(19) United States
(12) Patent Application Publication
(10) Pub. No.: US 2005/0250396 A1
(43) Pub. Date: Nov. 10, 2005

(54) Rescue lift

(76) Inventor: David H. Hayles, Stevenson Ranch, CA (US)

Correspondence Address: CHRISTIE, PARKER & HALE, LLP, PO BOX 7068, PASADENA, CA, 91109-7068, US

(21) Appl. No.: 10/841,352

(22) Filed: May 6, 2004

(51) Int. Cl. A61F 5/37

(52) U.S. Cl. 441/83; 244/137.2

(57) ABSTRACT

A compact rescue lift device adapted to be deployed from an elevated location, having a central spine with upper and lower portions, where the upper portion of the spine includes a means for coupling the device to a structure and the lower portion includes a plurality of seating surfaces which include both supporting and retaining portions, with the device also including a plurality of hand grips, where the device is adapted to carry multiple victims at a time.

### BACKGROUND OF THE INVENTION

The present invention relates to an emergency rescue lift device, and more specifically, to a compact rescue lift device adapted to be easily deployed from an elevated location, such as a helicopter or other aerial rescue vehicle or device.

In many emergency rescue situations, victims must be lifted or extracted from a particular, often dangerous location. A typical situation involves rescuing one or more victims from a body of water. For example, marine accidents or disasters often result in a helicopter being dispatched to the location of the emergency. Upon arrival at the site of the emergency, a rescuer is typically deployed from the helicopter. Victims are typically raised from the water into the helicopter in a “basket” type device, one victim at a time, requiring both the rescuer and additional victims to remain in the water for extended periods of time while each victim is lifted.

In addition to such marine rescues, severe weather, rains, and general storm conditions often cause floods, leaving victims of the circumstances stranded in a body of water. Such floods typically come somewhat unexpectedly, often trapping persons in a particular location, such as a vehicle. Such vehicles generally become immobilized, forcing the occupants to flee to the highest point to escape the floodwaters, typically the roof of the vehicle. Thus emergency rescues are often attempted to retrieve one or more victims from the roof of a partially submerged vehicle. Similar situations precipitate the need for rescues from the roof of buildings, also surrounded by water. In addition to floods, other circumstances such as fires, earthquakes, landslides or other natural disasters often result in victims being stranded in a remote or isolated location, where a rescue by air is the only feasible means of rescue. For example, a fire in a high rise building often leads to a number of victims fleeing to the roof of the building, where an aerial rescue, such as by a helicopter or crane, could then be attempted. In virtually all such circumstances, the more quickly such a rescue is accomplished, the greater the number of victims that will survive the emergency.

Therefore, the more quickly the helicopter is able to travel to the site of the emergency, and the more quickly the victims are able to be lifted or extracted, the more successful the rescue will be. A device that is compact enough to be stored within the helicopter will allow the helicopter to travel at maximum speed, without creating the additional drag of an outboard device. While the typical “basket” type rescue device may be stored within the helicopter, it still requires victims to be lifted one at a time, thereby lengthening the time require for extracting the victims. Thus there exists the need for a rescue lift device that is compact enough to be stored within a helicopter or other rescue vehicle, so as not to create additional drag on the vehicle, which does not add significant weight to the rescue vehicle, so as not to slow down the vehicle or diminish the capacity for rescue victims, and which is able to extract more than one victim at a time.

### SUMMARY OF THE INVENTION

The present invention is embodied in a compact rescue lift device that is adapted to be deployed from an elevated location and securely lift a plurality of victims at the same time.

One aspect of a particular embodiment of the present invention is that it is relatively compact, allowing for easy transportation within a rescue vehicle, and convenient storage when not in use.

Another aspect of particular embodiment of the present invention is that it is adapted to lift a plurality of victims at the same time.

Another aspect of a particular embodiment of the present invention is that it is universally adaptable to a variety of devices, and may be deployed from virtually any helicopter or crane, or other rescue vehicle or device, through a simple connection.

Another aspect of a particular embodiment of the present invention is that it provides means for victims to engage themselves with the lifting device.

Another aspect of a particular embodiment of the present invention is that it provides a secure lifting device for extracting victims from dangerous areas or situations.

Another aspect of a particular embodiment of the present invention is that it is adapted to allow a rescuer to secure the victims to the device.

Other features and advantages of the present invention will become apparent from the following description of the preferred embodiments, taken in conjunction with the accompanying drawings, which illustrate, by way of example, the principles of the invention.

### BRIEF DESCRIPTION OF THE DRAWINGS

FIG. 1 is a perspective view of one embodiment of the present invention; and

FIG. 2 is a perspective view of one embodiment of the present invention.

### DETAILED DESCRIPTION OF THE INVENTION

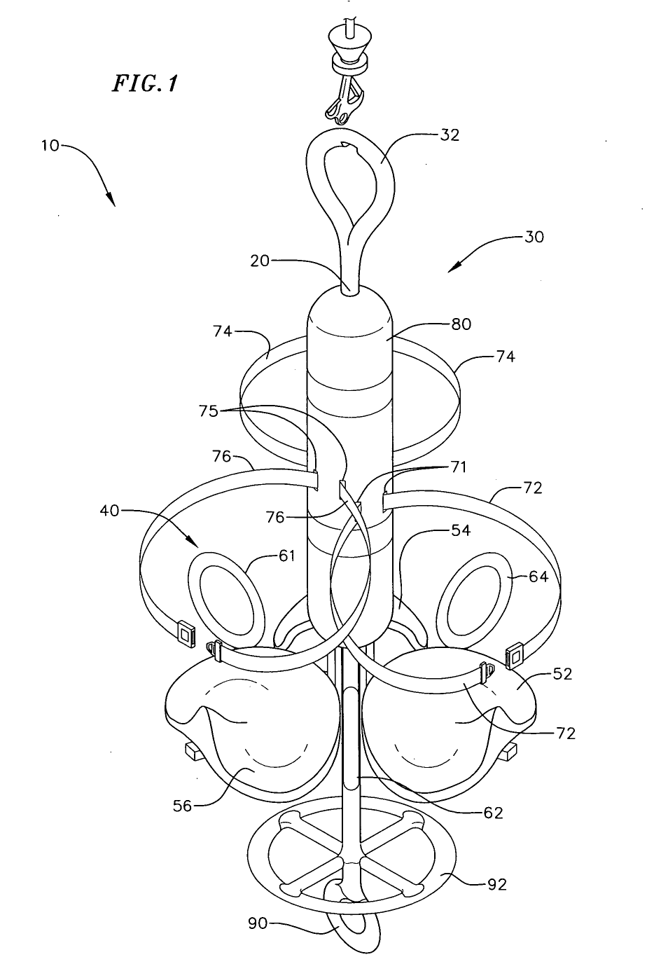

FIG. 1 illustrates a particular embodiment of the rescue lift device 10. The device includes a central spine 20. In one embodiment, the central spine 20 is substantially rigid, and constructed from aircraft grade aluminum. In another embodiment, the central spine 20 is constructed from stainless steel. It will be appreciated by those skilled in the art that in another embodiment, the central spine is constructed of titanium, and that in yet other embodiments, the central spine is constructed of a lightweight, high strength alloy. It will be further appreciated that in another embodiments, the central spine is non-metallic, and that in other embodiments, the central spine is of a substantially rigid material that is resistant to rust.

In one embodiment, the central spine 20 is of a substantially solid construction. In another embodiment, the central spine is of a substantially hollow construction. In another embodiment, the central spine is of a honeycomb-type construction. In another embodiment, the central spine is constructed of carbon fiber. It will be appreciated by those skilled in the art that in other embodiments, the central spine comprises a composite material. The central spine 20 includes an upper portion 30, and a lower portion 40. The upper portion 30 includes a means for connecting the rescue lift device to a lifting mechanism, such as a cable from a helicopter lifting arm. In one embodiment, the means for connection is a loop 32. In another embodiment, as shown in FIG. 1, the loop 32 is integral to the central spine 20. In yet another embodiment, the connection means is a hook. It will be appreciated by those skilled in the art that in other embodiments, the connection means is a threaded connector, a swivel connector, or any other coupling mechanism known in the art. In one embodiment, the inner upper surface of the loop 32 is machined to mate with the inner lower surface of a lifting hook. In such embodiment, for example, the mating surfaces of the loop 32 and the lifting hook are machined flat to provide a mating surface between the two, and to prevent rotation between the two.

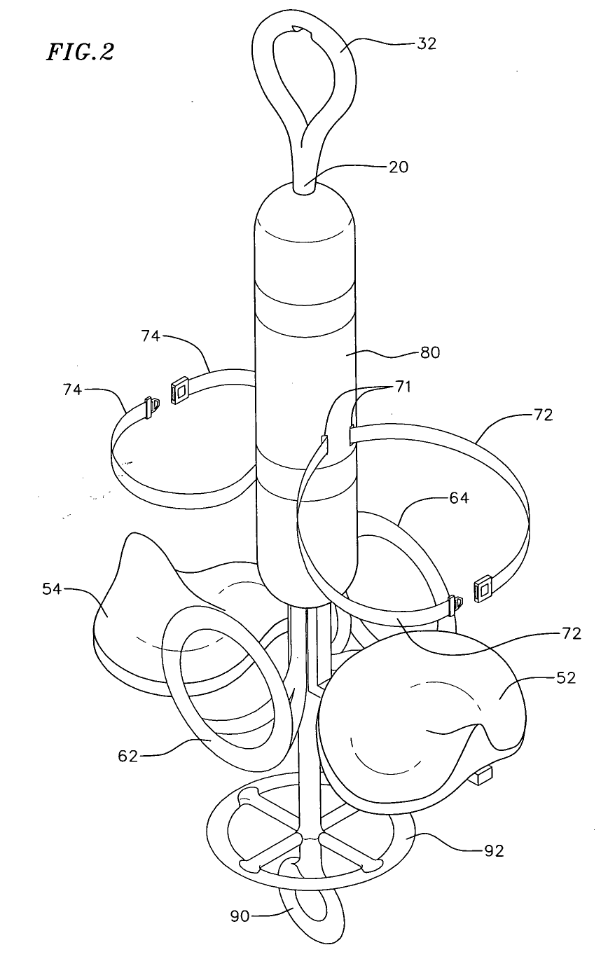

The lower portion 40 of the central spine 20 includes a plurality of seating surfaces 42, 44, 46. In one embodiment, as shown in FIG. 1, there are three seating surfaces, but it will be appreciated by those skilled in the art that in other embodiments, the number of seating surfaces is varied. In another embodiment, as shown in FIG. 2, there are two seating surfaces, while in another embodiment, there are four seating surfaces. In order to maintain consistency, corresponding features of the embodiments shown in FIGS. 1 and 2 have been annotated with like reference numerals.

In one embodiment, each seating surface 42, 44, 46 includes a supporting portion and a respective retaining portion 52, 54, 56. In one embodiment, this retaining portion extends upward from the seating surface, forming a shape similar to a saddle horn. This retaining portion acts to maintain victims in a seated position, and prevent them from falling from, or otherwise disengaging from, the seating surface.

In one embodiment, the respective supporting portions of the seating surfaces 42, 44, 46 are substantially horizontal, and extend horizontally from the lower portion 40 of the central spine 20. In another embodiment, the supporting portions of the seating surfaces are contoured into a slightly concave shape, to further aid in retaining victims on the seating surfaces. In one embodiment, the seating surfaces are constructed from the same material as the central spine. In another embodiment, the seating surfaces are constructed from a molded material, and coupled to the central spine. In one embodiment, the seating surfaces are constructed of aluminum. In another embodiment, the seating surfaces are constructed substantially of a molded plastic material. It will be appreciated by those skilled in the art that in other embodiments, the seating surfaces are constructed of a combination of materials, and that any materials known to those skilled in the art may be used to achieve the particular shape of the seating surfaces without departing from the invention.

In another embodiment, at least a portion of the central spine 20 is covered by a cushioning material 80. In one embodiment, the cushioning material is buoyant. In another embodiment, the cushioning material is an air-filled buoyant material. In such embodiments, the amount of cushioning material, and/or the degree to which the material is inflated are adjusted to achieve the desired overall buoyancy of the device.

In the embodiment shown in FIG. 1, adjacent the seating surfaces are hand grips 62, 64, 66. It will be appreciated that in other embodiments, the number of hand grips may be varied, and that in other embodiments, no hand grips are provided. In yet another embodiment, the hand grips are defined by a portion of the seating surface. The hand grips provide a surface for victims to hold, and also provide an attachment point for accessories, such as additional ropes or hooks. In addition, the hand grips provide an easy engagement point for victims to take hold of the device. Once a victim has engaged a hand grip, he or she may use the hand grip to pull him or herself onto the seating surface. Once on the seating surface, the retaining portion of the seating surface will maintain the victim on the seating surface, and the hand grip will provide an additional surface for the victim to hold. In one embodiment, as shown in FIG. 1, the hand grips are circular, and are oriented substantially parallel to the seating surfaces, extending radially outward from central spine. In another embodiment, as shown in FIG. 2, the hand grips are substantially parallel to one another. It will be appreciated by those skilled in the art that in other embodiments, the orientation of the hand grips is varied, from being substantially parallel to the seating surfaces to being substantially perpendicular to the seating surfaces, and from being in a substantially vertical orientation to a substantially horizontal orientation. It will be further appreciated by those skilled in the art that in other embodiments, the shape of the hand grips is straight, curved, circular, or a combination of such shapes.

In one embodiment, the buoyancy of the device is adjusted so that when deployed, unloaded (not carrying any victims), the lower portion 40 of the central spine 20 is at least partially submerged. In this embodiment, the seating surfaces 42, 44, 46 are also at least partially submerged, allowing victims to more easily get onto the device, as they or the rescuer may use the buoyancy of the water to more easily engage the device. In one embodiment, buoyant material is coupled to the seating surfaces 42, 44, 46 to achieve a desired overall buoyancy for the device. In one embodiment, the seating surfaces 42, 44, 46 of the device contain buoyant material. In another embodiment, the seating surfaces themselves are buoyant.

In one embodiment, restraints 72, 74, 76 are provided to further secure the victims, and to maintain them in the seating surfaces while the device is lifted. In one embodiment, the respective restraints 72, 74, 76 are coupled to the central spine 20 through slots 71, 73, 75 in the cushioning material. In another embodiment, restraints are coupled to the seating surfaces. In another embodiment, restraints are coupled to the central spine and to the seating surfaces. In yet another embodiment, restraints extend from the central spine, over the shoulders of the victims, and couple to the respective restraining portions of the seating surfaces. In one embodiment, the restraints are adapted to be engaged with one hand, allowing a rescuer to more easily assist and secure a victim on a seating surface. It will be appreciated by those skilled in the art that in one embodiment, the restraints utilize a “seat belt” type coupling, while in other embodiments the restraints comprise continuous closed-loop portions which may be fit over or around victims and then tightened. It will be further appreciated by those skilled in the art that in other embodiments, a variety of restraint configurations are used. In one embodiment, rather than coupling two portion of the restraint together in a “seat belt” type coupling, the restraint is fixedly attached to the device at one end, and removably attached to the device at the other end, to enable a rescuer to attach and detach the restraint with one hand. In one embodiment, the restraint is attached to the center spine. In another embodiment, the restraint is attached to the seating surface. In another embodiment, the restraint is fixedly attached to a first hand grip, and removably attached to a second hand grip, so that the restraint extends across a seating surface. In yet another embodiment, the device is attached to at least two of: the central spine, the seating surfaces, and the hand grips. In another embodiment, the restraint is retractable. A retractor portion houses the restraint, and is fixedly attached to the device. A coupling portion is fixedly attached to a second point on the device. The restraint is extended from the retractor portion, around a victim, and is removably attached to the coupling portion. When not in use, the restraint is retracted back into the retractor portion.

In one embodiment, a lower loop extends from the lower portion 40 of the central spine. This loop provides an additional attachment point for the device, and further provides an additional point for rescuers or victims to engage the device. In one embodiment, the lower loop is configured in a substantially vertical orientation. In another embodiment, the lower loop is configured in a substantially horizontal orientation. In this embodiment, the lower loop may be used as a step to assist in engaging and boarding the device. The lower loop also provides a foot rest for those on the device while being lifted, giving victims an additional surface to engage and an increased sense of security. In one embodiment, the device includes a substantially horizontal loop 92 defining a step/foot rest, and a substantially vertical loop 90 defining an attachment point. In another embodiment, the lower loop is telescopically coupled to the lower portion of the central spine, such that it is retracted while stored, and extended while deployed. In one embodiment, the lower loop 90 contains buoyant material sufficient to achieve a particular overall buoyancy of the device. In one embodiment, loop 92 contains buoyant material sufficient to achieve a particular overall buoyancy of the device.

In one embodiment, the rescue lift device is approximately four feet high, and approximately three feet in diameter, allowing it to be transported within a U.S. Coast Guard HH-65 helicopter, and deployed from the standard lifting arm. However, it will be appreciated by those skilled in the art that in other embodiments, the dimensions may be varied, depending upon the specific application, and the specific helicopter or other vehicle in which the device is designed to fit. It will be further appreciated, by those skilled in the art that many of the advantages of the present invention still exist in an embodiment to be transported outside of a rescue vehicle, such as a helicopter.

Although the invention has been described in detail with reference only to the preferred embodiments, those having ordinary skill in the art will appreciate that various modifications, including modifications to shape and size, can be made without departing from the spirit and scope of the invention. Accordingly, the invention is defined with reference to the following claims.

1. A rescue lift device, adapted to be deployed from a an elevated location, comprising:
a substantially rigid central spine, having an upper portion, and a lower portion;
a means for connecting the rescue lift device to a structure, said means coupled to the upper portion of the central spine;
a plurality of seating surfaces, coupled to the lower portion of the central spine, each seating surface having a substantially horizontal supporting portion and a retaining portion extending substantially upward therefrom; and
a plurality of hand grips, coupled to the lift device.

2. A rescue lift device as in claim 1, wherein the elevated location is a helicopter.

3. A rescue lift device as in claim 1, wherein the seating surfaces comprise molded shapes.

4. A rescue lift device as in claim 1, wherein the hand grips are integrated into the seating surfaces.

5. A rescue lift device as in claim 1, wherein the seating surfaces are arranged radially about the central spine.

6. A rescue lift device as in claim 5, wherein the hand grips are located between adjacent seating surfaces.

7. A rescue lift device as in claim 1, wherein the hand grips are circular in shape.

8. A rescue lift device as in claim 1, wherein the hand grips are elliptical in shape.

9. A rescue lift device as in claim 1, further comprising a pad covering at least a portion of the central spine.

10. A rescue lift device as in claim 9, wherein the pad is buoyant.

11. A rescue lift device as in claim 1, wherein the means for connecting the device to a structure is a loop.

12. A rescue lift device as in claim 11, wherein the loop includes a flat portion, adapted to mate with a flat portion of a connecting hook.

13. A rescue lift device as in claim 11, wherein the loop is integral to the central spine.

14. A rescue lift device as in claim 1, further comprising a restraint coupled to the device.

15. A rescue lift device as in claim 14, wherein the restraint is retractable.

16. A rescue lift device as in claim 14, wherein the restraint is coupled to at least two of the central spine, a seating surface, and a hand grip.

17. A rescue lift device as in claim 1, further comprising a lower loop coupled to the central spine.

18. A rescue lift device as in claim 17, wherein the lower loop is oriented in a substantially vertical configuration.

19. A rescue lift device as in claim 17, wherein the lower loop is oriented in a substantially horizontal configuration.

20. A rescue lift device, adapted to be deployed from a an elevated location, comprising:
a substantially rigid central spine having an upper portion and a lower portion;
a connection loop coupled to the upper portion of the central spine;
a plurality of seating surfaces coupled to the lower portion of the central spine, each seating surface having a substantially horizontal supporting portion and a retaining portion extending substantially upward therefrom;
a plurality of hand grips coupled to the lift device;
a plurality of restraints coupled to the lift device; and
a lower loop portion coupled to the lower portion of the central spine.
